(19) United States
(12) Patent Application Publication
(10) Pub. No.: US 2023/0404781 A1
(43) Pub. Date: Dec. 21, 2023

(54) IN-VIVO INDWELLING STENT AND STENT DELIVERY SYSTEM

(71) Applicant: TERUMO KABUSHIKI KAISHA, Tokyo (JP)

(72) Inventors: Yukiko TANABE, Ashigarakami-gun (JP); Takashi KITAOKA, Ashigarakami-gun (JP); Taishi NIIMI, Ashigarakami-gun (JP); Hiroshi YAMAGUCHI, Ashigarakami-gun (JP)

(73) Assignee: TERUMO KABUSHIKI KAISHA, Tokyo (JP)

(21) Appl. No.: 18/461,632

(22) Filed: Sep. 6, 2023

Related U.S. Application Data
(63) Continuation of application No. PCT/JP2021/048279, filed on Dec. 24, 2021.

(30) Foreign Application Priority Data
Mar. 29, 2021 (JP) 2021-056267

(51) Int. Cl. A61F 2/90; A61F 2/966

(52) U.S. Cl. CPC A61F 2/90; A61F 2/966

(57) ABSTRACT

A stent includes a plurality of annular bodies formed with linear components, and connection portions. The connection portions being first pattern connection portions that connect one end side apexes and connection linear portions located closer to one end side than the one end side apexes or second pattern connection portions that connect the connection linear portions and the connection linear portions closer to the other end side than the connection linear portions, and other connection portions selected from the first or second pattern connection portions which are not selected, third pattern connection portions that connect the other end side apexes and the connection linear portions closer to the other end side than the other end side apexes, and fourth pattern connection portions that connect the one end side apexes and the other end side apexes located closer to one end side than the one end side apexes.

### CROSS-REFERENCES TO RELATED APPLICATIONS

This application is a continuation of International Application No. PCT/JP2021/048279 filed on Dec. 24, 2021, which claims priority to Japanese Application No. 2021-056267 filed on Mar. 29, 2021, the entire content of both of which is incorporated herein by reference.

### TECHNOLOGICAL FIELD

The present disclosure generally relates to an in-vivo indwelling stent and a stent delivery system to be used for improving stenosis or occlusion generated in a body lumen such as a blood vessel, a bile duct, a trachea, an esophagus, or a urethra.

### BACKGROUND DISCUSSION

An in-vivo indwelling stent is used for treating various diseases caused by stenosis or occlusion of a blood vessel or other lumens in a living body. The stent is formed in a tubular shape to expand the stenotic or occluded site and secure an expanded inner diameter at the stenotic or occluded site.

The stent has a small diameter at the time of insertion so as to be inserted into the body from outside the body, expands at a target stenosis or occlusion site to increase the diameter, and holds a lumen in the expanded inner diameter as it is.

As the stent, a metal wire material or a cylindrical stent obtained by processing a metal tube is generally used. The stent is attached to a catheter, or the like, in a thinned state, inserted into a living body, expanded by some method at a target site, and fixed in close contact with an inner wall of a lumen to maintain a shape of the lumen.

Stents are distinguished into self-expanding stents and balloon-expanding stents by functions and indwelling methods. A balloon-expanding stent itself has no expansion function. After the stent mounted on the balloon is inserted into a target site, the balloon is expanded, and the stent is expanded (plastically deformed) by expansion force of the balloon and fixed in close contact with an inner surface of a target lumen. The balloon-expanding type of stent requires work of expanding the stent as described above. On the other hand, a self-expanding stent has an expansion function in the stent itself and is inserted into a living body in a thin and shrunk state, opened at a target site to return to its original expanded state and fixed in close contact with an inner wall of a lumen to maintain a shape of the lumen.

One of causes of an ischemic cerebral artery disorder is stenosis/occlusion of the intracranial artery. As a general treatment method, the risk can be reduced by antiplatelet therapy. However, medical treatment is also limited, and a patient exhibiting drug resistance is treated by balloon expansion or stent indwelling. In stent indwelling, it is a problem that there are many perioperative complications, and one of the factors is in-stent thrombogenesis. The brain is said to have poor drug accessibility due to a blood-brain barrier, and considered to be an environment in which complications due to thrombus formation in the stent are likely to occur even if antithrombotic therapy is continued after surgery.

The intracranial blood vessels include many intricately snaking portions. In a case where the stent is indwelled at a bent portion, if the stent has poor followability, buckling occurs such that stent struts enter a lumen of the blood vessel, inhibits blood flow, thereby causing thrombus. Also, the stent struts can damage a wall of the blood vessel and cause perforation or injury. To achieve a stent with a reduced risk of in-stent thrombosis, it is necessary to devise the stent to ensure followability without damaging blood vessels.

As a self-expanding stent, a stent disclosed in Japanese Patent Application Publication No. 2003-93519 A has been proposed.

The stent disclosed in Japanese Patent Application Publication No. 2003-93519 A includes a plurality of wavy struts extending in an axial direction from one end side to the other end side of the stent and arranged in a circumferential direction of the stent, and a plurality of connection struts that connect the adjacent wavy struts and extend in a predetermined long axis direction, and an end portion of a wavy strut is coupled to an end portion of a wavy strut in the vicinity.

However, in Japanese Patent Application Publication No. 2003-93519 A, a plurality of wavy struts extending in the axial direction from one end side to the other end side of the stent and arranged in the circumferential direction of the stent are provided. As a result of research, it has been found that the stent of Japanese Patent Application Publication No. 2003-93519 A has insufficient expansion force and followability because the wavy struts extend in the axial direction.

### SUMMARY

An in-vivo indwelling stent is disclosed, which is a tubular in-vivo indwelling stent in which annular bodies formed with wavy and circular linear components including a plurality of one end side apexes on one end side in an axial direction and a plurality of other end side apexes on the other end side in the axial direction of the stent are arranged in a plurality of axial directions, and adjacent annular bodies are connected by connection portions, and which has favorable storability and followability by radial compression and sufficient expansion force, and a stent delivery system.

In accordance with an aspect, an in-vivo indwelling stent in which a plurality of annular bodies formed in an annular (or a circular shape) with linear components are arranged in an axial direction, and adjacent annular bodies are connected by connection portions, each of the annular bodies being formed with one endless linear component having a plurality of one end side apexes located on one end side in the axial direction of the stent, a plurality of other end side apexes located on the other end side in the axial direction of the stent, and a plurality of connection linear portions that connect the one end side apexes and the other end side apexes, and adjacent annular bodies of a plurality of sets located in at least a central portion of the stent being connected by a plurality of connection portions, and 25% to 75% of a total number of the connection portions located in the central portion being basic pattern connection portions including first pattern connection portions that connect the one end side apexes and the connection linear portions located closer to one end side than the one end side apexes or second pattern connection portions that connect the connection linear portions and the connection linear portions located closer to the other end side than the connection linear portions, and remaining connection portions located in the central portion being pattern connection portions of at least one type selected from the first pattern connection portions or the second pattern connection portions which are not selected in the basic pattern connection portions, third pattern connection portions that connect the other end side apexes and the connection linear portions located closer to the other end side than the other end side apexes, and fourth pattern connection portions that connect the one end side apexes and the other end side apexes located closer to one end side than the one end side apexes, and the number of connection portions that connect the adjacent annular bodies located in the central portion being the same as, larger by one than, or smaller by one than the number of the one end side apexes or the number of the other end side apexes of the annular bodies connected by the connection portions.

A stent delivery system including: a sheath; the stent stored in a distal end portion of the sheath; and a shaft that is to be inserted into the sheath and releases the stent from a distal end of the sheath, in which the stent is formed in a substantially cylindrical shape, compressed in a central axis direction at the time of in-vivo insertion, and expands outward to restore a shape before compression at the time of in-vivo indwelling.

In accordance with another aspect, an in-vivo indwelling stent comprising: a plurality of annular bodies formed in a circular shape with linear components arranged in an axial direction, and adjacent annular bodies of the plurality of annual bodies connected by connection portions; each of the plurality of annular bodies being formed with one endless linear component having a plurality of one end side apexes located on one end side in the axial direction of the stent, a plurality of other end side apexes located on the other end side in the axial direction of the stent, and a plurality of connection linear portions that connect the one end side apexes and the other end side apexes; and the adjacent annular bodies including at least a central portion of the stent being connected by a plurality of connection portions, the plurality of connection portions located in the central portion being basic pattern connection portions including first pattern connection portions that connect the one end side apexes and the connection linear portions located closer to one end side than the one end side apexes or second pattern connection portions that connect the connection linear portions and the connection linear portions located closer to the other end side than the connection linear portions, and remaining connection portions of the plurality of connection portions located in the central portion being pattern connection portions of at least one type selected from the first pattern connection portions or the second pattern connection portions which are not selected in the basic pattern connection portions, third pattern connection portions that connect the other end side apexes and the connection linear portions located closer to the other end side than the other end side apexes, and fourth pattern connection portions that connect the one end side apexes and the other end side apexes located closer to one end side than the one end side apexes, and the number of connection portions that connect the adjacent annular bodies located in the central portion being the same as, larger by one than, or smaller by one than the number of the one end side apexes or the number of the other end side apexes of the annular bodies connected by the connection portions.

In accordance with an aspect, an in-vivo indwelling stent comprising: a plurality of annular bodies formed with linear components, each of the plurality of annular bodies being formed with one endless linear component having a plurality of one end side apexes located on one end side in the axial direction of the stent, a plurality of other end side apexes located on the other end side in the axial direction of the stent, and a plurality of connection linear portions that connect the one end side apexes and the other end side apexes; a plurality of connection portions, the plurality of connection portions including basic pattern connection portions and other connection portions, the basic pattern connection including first pattern connection portions that connect one end side apexes and connection linear portions located closer to one end side than the one end side apexes or second pattern connection portions that connect the connection linear portions and the connection linear portions closer to the other end side than the connection linear portions, and the other connection portions being selected from the first or second pattern connection portions which are not selected, third pattern connection portions that connect the other end side apexes and the connection linear portions closer to the other end side than the other end side apexes, and fourth pattern connection portions that connect the one end side apexes and the other end side apexes located closer to one end side than the one end side apexes; and wherein a number of the connection portions between adjacent annular bodies is the same as, larger by one than, or smaller by one than the number of the one end side apexes or the other end side apexes of the annular bodies to be connected.

### BRIEF DESCRIPTION OF THE DRAWINGS

FIG. 1 is a developed view at the time of manufacturing an in-vivo indwelling stent according to an embodiment of the present disclosure.

FIG. 2 is a partially enlarged view of the in-vivo indwelling stent of FIG. 1.

FIG. 3 is a developed view at the time of manufacturing an in-vivo indwelling stent according to another embodiment of the present disclosure.

FIG. 4 is a partially enlarged view of the in-vivo indwelling stent of FIG. 3.

FIG. 5 is a developed view at the time of manufacturing an in-vivo indwelling stent according to another embodiment of the present disclosure.

FIG. 6 is a partially enlarged view of the in-vivo indwelling stent of FIG. 5.

FIG. 7 is a developed view at the time of manufacturing an in-vivo indwelling stent according to another embodiment of the present disclosure.

FIG. 8 is a partially enlarged view of the in-vivo indwelling stent of FIG. 7.

FIG. 9 is a developed view at the time of manufacturing an in-vivo indwelling stent according to another embodiment of the present disclosure.

FIG. 10 is a partially enlarged view of the in-vivo indwelling stent of FIG. 9.

FIG. 11 is a front view of a stent delivery system of an embodiment of the present disclosure.

FIG. 12 is a partially broken enlarged view of a distal end portion of the stent delivery system illustrated in FIG. 11.

### DETAILED DESCRIPTION

Set forth below with reference to the accompanying drawings is a detailed description of embodiments of an in-vivo indwelling stent.

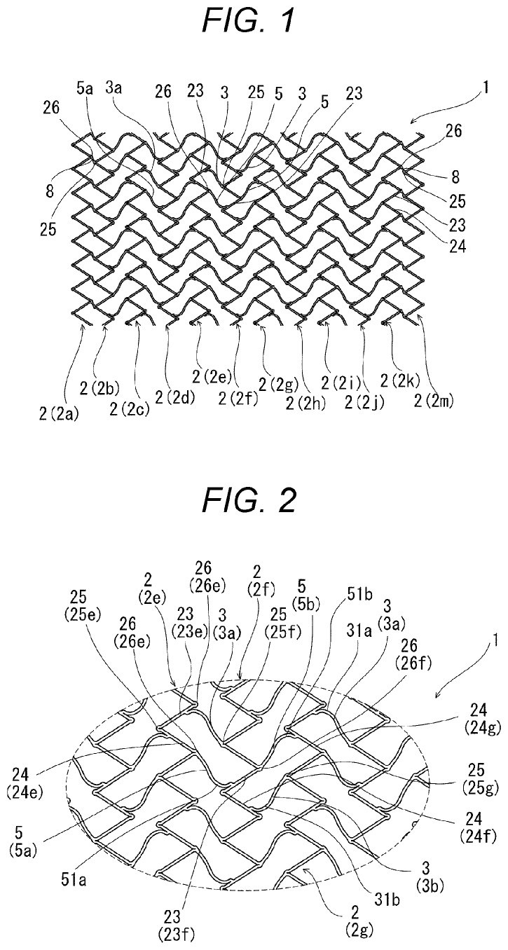

As illustrated in FIGS. 1 and 2, an in-vivo indwelling stent 1 of the present disclosure is an in-vivo indwelling stent in which a plurality of annular bodies 2 each formed in an annular shape (or a circular shape) with one endless linear component (strut) are arranged in an axial direction, and adjacent annular bodies 2 (2a, 2b, 2c, 2d, 2e, 2f, 2g, 2h, 2i, 2j, 2k, 2m) are connected by connection portions 3 and 8. Note that in FIGS. 1 and 2, a left side is referred to as “one end side”, and a right side is referred to as “the other end side”.

Each of the annular bodies 2 (2a, 2b, 2c, 2d, 2e, 2f, 2g, 2h, 2i, 2j, 2k, 2m) is formed with one endless linear component having a plurality of one end side apexes 25 (one end side bent portions) located on one end side in the axial direction of the stent, a plurality of other end side apexes 26 (other end side bent portions) located on the other end side in the axial direction of the stent, and a plurality of connection linear portions 23, 24 that connect the one end side apexes 25 and the other end side apexes 26.

A plurality of sets of adjacent annular bodies 2 located at least in a central portion (i.e., a portion between the one end side of the stent and the other end side of the stent) of the stent are connected by a plurality of connection portions. Further, for example, 25% to 75% of a total number of the connection portions located in the central portion are basic pattern connection portions including first pattern connection portions 3 that connect the one end side apexes 25 and the connection linear portions 23 or 24 located closer to one end side than the one end side apexes 25, or second pattern connection portions 4 that connect the connection linear portions 23 or 24 and the connection linear portions 23 or 24 located closer to the other end side than the connection linear portions 23 or 24. Further, the remaining connection portions located in the central portion are pattern connection portions of at least one type selected from the first pattern connection portions 3 or the second pattern connection portions 4 which are not selected in the basic pattern connection portions, third pattern connection portions 5 that connect the other end side apexes 26 and the connection linear portions 23 or 24 located closer to the other end side than the other end side apexes 26, and fourth pattern connection portions 6 and 7 that connect the one end side apexes 25 and the other end side apexes 26 located closer to the one end side than the one end side apexes 25.

Furthermore, the number of connection portions that connect the adjacent annular bodies 2 located in the central portion is the same as or larger by one or smaller by one than the number of the one end side apexes or the number of the other end side apexes of the annular bodies connected by the connection portions. Preferably, the number of connection portions is the same as the number of the one end side apexes or the number of the other end side apexes of the annular bodies connected by the connection portions. The phrase “larger by one than the number of the one end side apexes or the number of the other end side apexes” refers to a state in which a connection portion is added between any connection portions adjacent to each other in a circumferential direction of the stent. The pattern of the connection portion to be added may be the connection portion of any pattern described above. In addition, smaller by one than the number of the one end side apexes or the number of the other end side apexes refers to a state in which one connection portion that connects adjacent annular bodies 2 located at the central portion is thinned out (or reduced) in embodiments illustrated in the drawings.

The stent 1 can be effectively used as a stent for expansion of a cerebral artery (stent for treatment of cerebral artery stenosis).

The stent 1 of this embodiment is a so-called self-expanding stent that is formed in a substantially cylindrical shape, is compressed in a central axis direction at the time of in-vivo insertion and expands outward to restore a shape before compression at the time of in-vivo indwelling.

As illustrated in FIGS. 1 and 2, the stent 1 of this embodiment has a form in which a plurality of annular bodies 2 (2a, 2b, 2c, 2d, 2e, 2f, 2g, 2h, 2i, 2j, 2k, 2m) are arranged in parallel in the axial direction so as to be adjacent to each other in the axial direction, and the adjacent annular bodies are connected. Each of the annular bodies 2 (2a, 2b, 2c, 2d, 2e, 2f, 2g, 2h, 2i, 2j, 2k, 2m) is formed with a wavy and annular linear component including a plurality of one end side bent portions having apexes 25 on one end side in the axial direction and a plurality of other end side bent portions having apexes 26 on the other end side in the axial direction of the stent 1. The one end side bent portions and the other end side bent portions are connected by the connection linear portions 23 and 24. In the stent of this embodiment, a shape of the connection linear portions 23 and 24 is substantially a straight line or a gentle curve.

The number of the annular bodies 2 in the stent of the present invention can be, for example, preferably 4 to 100, and more preferably 6 to 80. The number of annular bodies 2 in the stents 1 to 1d can be, for example, 12 or 14.

As illustrated in FIG. 1, the annular body 2 (2a, 2b, 2c, 2d, 2e, 2f, 2g, 2h, 2i, 2j, 2k, 2m) in the stent 1 of this embodiment includes a plurality of one end side apexes 25 and other end side apexes 26 having substantially the same pitch. The number of the one end side apexes 25 and the other end side apexes 26 in one annular body of the stent of the present invention can be, for example, preferably 4 to 12, and more preferably 4 to 10.

First, the stent 1 of the embodiment illustrated in FIGS. 1 and 2 will be described.

In the stent 1 of this embodiment, as illustrated in FIGS. 1 and 2, between a plurality of sets of adjacent annular bodies 2 (2b, 2c, 2d, 2e, 2f, 2g, 2h, 2i, 2j, 2k) located in the central portion, first pattern connection portions 3 (3a, 3b) that connect the one end side apexes 25 (for example, 25f, 25g) and the connection linear portions 23 or 24 (for example, 23e and 24f) located closer to one end side than the one end side apexes 25 (for example, 25f, 25g) are set as a basic pattern, and third pattern connection portions 5 (5a, 5b) that connect the other end side apexes 26 (for example, 26e, 26f) and the connection linear portions 23 or 24 (for example, 23f, 24g) located closer to the other end side than the other end side apexes 26 (for example, 26e, 26f) are provided between annular bodies connected by the first pattern connection portions 3 (3a, 3b).

Advantages of the first pattern connection portions 3 and the third pattern connection portions 5 are suppression of buckling in a bent blood vessel and enabling following the blood vessel in a state where the lumen is held. The connection portions connected to the connection linear portions 23, 24 can deform compressive force in the axial direction applied to the stent at a bent portion of the blood vessel so as to act in a rotational direction, so that buckling can be suppressed.

In general, in a case where a stent is indwelled in a bent portion, compressive force in the axial direction is applied so that adjacent annular members approach each other inside the bent portion. The compressive force is transmitted to the connection portion that connects the annular members, and the stent is deformed. However, in a case where the compressive force cannot be relaxed only by deformation of the connection portion and a peripheral strut, buckling in which the stent is greatly deformed can occur. In a case where the stent is buckled, the stent enters the lumen of the blood vessel, so that a diameter of the blood vessel cannot be maintained at that portion, and the stent becomes an environment (or a hotbed) for thrombus formation.

In a case where the connection portion is formed as an apex, compressive force due to indwelling of the bent portion acts in the axial direction, but in a case where the connection portion is formed as a connection linear portion, the compressive force changes from the axial direction to the rotational direction, so that buckling can be suppressed.

In the stent 1 of this embodiment, all the adjacent annular bodies located in the central portion are connected by the first pattern connection portions 3 that connect the one end side apexes 25 and the connection linear portions 23 or 24 located closer to one end side than the one end side apexes 25 and the third pattern connection portions 5 that connect the other end side apexes 26 and the connection linear portions 23 or 24 located closer to the other end side than the other end side apexes 26, and the first pattern connection portions 3 and the third pattern connection portions 5 are alternately arranged in the circumferential direction between the adjacent annular bodies.

In the stent 1 of this embodiment, the adjacent annular bodies 2 (2b, 2c, 2d, 2e, 2f, 2g, 2h, 2i, 2j, 2k) of a plurality of sets located in the central portion are connected only by the first pattern connection portions 3 (3a, 3b) and the third pattern connection portions 5 (5a, 5b). Thus, a ratio of each of the first pattern connection portions 3 (3a, 3b) and the third pattern connection portions 5 (5a, 5b) can be, for example, 50%. As in this embodiment, the adjacent annular bodies 2 located in the central portion are preferably connected by connection portions of different patterns.

In the stent 1 of this embodiment, the first pattern connection portions 3 (3a, 3b) and the third pattern connection portions 5 (5a, 5b) are alternately arranged in the circumferential direction and inclined in the same circumferential direction.

In addition, it is preferable that the connection portions adjacent to each other in the axial direction of the stent in all the adjacent annular bodies located in the central portion extend in different circumferential directions. Furthermore, it is preferable that the connection portions of the same pattern adjacent to each other in the axial direction of the stent positioned in the central portion extend in different circumferential directions.

In the stent 1 of this embodiment, the first pattern connection portion 3a and the first pattern connection portion 3b adjacent to each other in the axial direction of the stent 1 are inclined in different circumferential directions, and similarly, the third pattern connection portion 5a and the third pattern connection portion 5b adjacent to each other in the axial direction of the stent 1 are also inclined in different circumferential directions. Furthermore, in the stent 1 of this embodiment, the first pattern connection portion 3 (3a, 3b) and the third pattern connection portion 5 (5a, 5b) have substantially the same axial length.

Furthermore, in the stent 1 of this embodiment, one end of the first pattern connection portion 3 (3a, 3b) is connected to the other end side bent portion side (the other end side apex side) of the connection linear portion 23 or 24 (for example, 23e, 24f). Specifically, one end of the first pattern connection portion 3 (3a, 3b) is connected to a position closer to the other end side apex than the central portion of the connection linear portion 23 or 24 (for example, 23e, 24f). The other end of the third pattern connection portion 5 (5a, 5b) is connected to one end side bent portion side (one end side apex side) of the connection linear portion 23 or 24 (for example, 23f, 24g). Specifically, the other end of the third pattern connection portion 5 (5a, 5b) is connected to a position closer to the one end side apex than the central portion of the connection linear portion 23 or 24 (for example, 23f, 24g).

In the stent 1 of this embodiment, the first pattern connection portion 3a includes a one end curved portion 31a curved in a direction opposite to the inclination direction of the connection portion 3a (upward at the right-downward connection portion in FIG. 2). An apex of the curved portion 31a is close to the other end side apex 26 of the connection linear portion to which the connection portion 3a is connected. The first pattern connection portion 3b includes a one end curved portion 31b curved in a direction opposite to the inclination direction of the connection portion (downward at the right-upward connection portion in FIG. 2). An apex of the curved portion 31b is close to the other end side apex 26 of the connection linear portion to which the connection portion 3b is connected.

In the stent 1 of this embodiment, the third pattern connection portion 5a includes an other end portion curved portion 51a curved in the inclination direction of the connection portion 5a (downward at the right-downward connection portion in FIG. 2). An apex of the other end portion curved portion 51a of the third pattern connection portion 5a is close to the one end side apex 25 of the connection linear portion to which the connection portion 5a is connected.

The third pattern connection portion 5b includes an other end portion curved portion 51b curved in the inclination direction of the connection portion 5b (upward at the right-upward connection portion in FIG. 2). An apex of the other end curved portion 51a of the third pattern connection portion 5b is close to the one end side apex 25 of the connection linear portion to which the connection portion 5b is connected.

Then, in the stent 1 of this embodiment, an outer surface of the stent 1 has a plurality of convex portions generated by intermediate portions of the first pattern connection portions 3 and the third pattern connection portions 5 protruding outward. Specifically, the first pattern connection portions 3 (3a, 3b) and the third pattern connection portions 5 (5a, 5b) protrude outward at the time of expansion of the stent and form convex portions with a protrusion amount increasing from both ends of the first pattern connection portions 3 and the third pattern connection portions 5 toward the central portion, on the outer surface of the stent. A height of the convex portions can be, for example, preferably equal to or greater than 50 μm. A distance between the convex portions in the axial direction is substantially the same as a distance between the one end side apexes (distance between the other end side apexes) of the adjacent annular bodies.

In the stent 1 of this embodiment, between the adjacent annular bodies 2, substantially rectangular closed cells formed by the connection linear portions 23 and 24 of one annular body 2, the connection linear portions 23 and 24 of the other adjacent annular body 2, the first pattern connection portion 3 and the third pattern connection portion 5 adjacent to each other in the circumferential direction and inclined in a first circumferential direction (first direction) of the stent are arranged in parallel in the circumferential direction. Further, substantially rectangular closed cells inclined in a second direction opposite to the first circumferential direction of the stent 1 are arranged in parallel in the circumferential direction adjacent in the axial direction to the substantially rectangular closed cells. Furthermore, in the stent 1 of this embodiment, one end portions of the substantially rectangular closed cells inclined in the second direction enter recesses between the other ends of the substantially rectangular closed cells inclined in the first direction. Thus, favorable expansion retention force is provided.

Furthermore, as illustrated in FIG. 1, the stent 1 of this embodiment includes: a one end portion annular body connection portion 8 that connects the other end side apex 26 of the annular body 2a positioned at one end and the one end side apex 25 of the annular body 2b adjacent to the annular body 2a positioned at one end; and an other end portion annular body connection portion 8 that connects the one end side apex 25 of the annular body 2m positioned at the other end and the other end side apex 26 of the annular body 2k adjacent to the annular body 2m positioned at the other end. The connection portion 8 has a shorter axial length of the stent than the connection portion 3 and the connection portion 5. Thus, in the stent 1 of this embodiment, so-called closed cells having a substantially rhombic shape are provided at both ends, so that the stent 1 has relatively high axial rigidity.

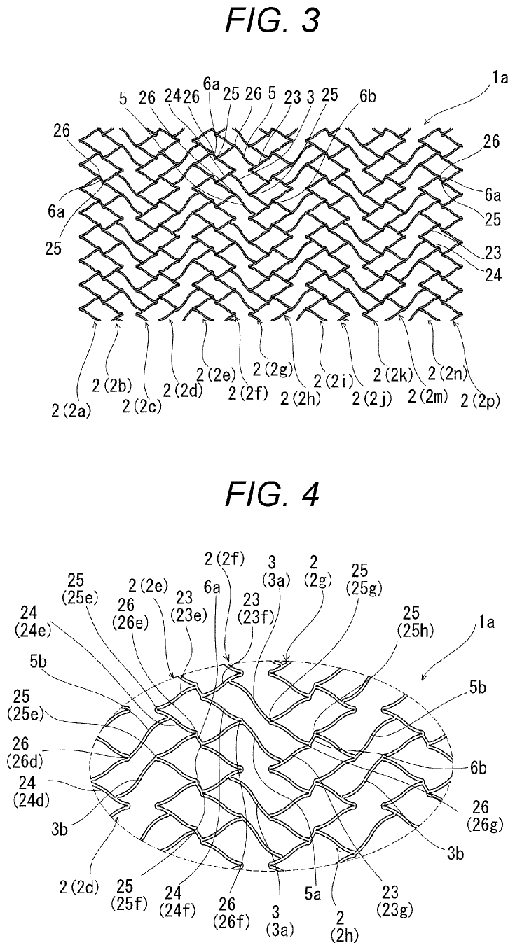

Next, a stent 1a of the embodiment illustrated in FIGS. 3 and 4 will be described.

In the stent 1a of this embodiment, as illustrated in FIGS. 3 and 4, the adjacent annular bodies 2 of a plurality of sets (first sets) located in the central portion (2b and 2c, 2d and 2e, 2f and 2g, 2h and 2i, 2j and 2k, 2m and 2n) are further connected by the third pattern connection portions 5 (5a, 5b) that connect the other end side apexes 26 (for example, 26f, 26d) and the connection linear portions 23 or 24 (for example, 23g, 24e) located closer to the other end side than the other end side apexes 26 (for example, 26f, 26d) as a basic pattern, and further by the first pattern connection portions 3 (3a, 3b) that connect the one end side apexes 25 (for example, 25g, 25e) and the connection linear portions 23 or 24 (for example, 23f, 24d) located closer to one end side than the one end side apexes 25 (for example, 25g, 25e).

In the stent 1a of this embodiment, the adjacent annular bodies of a plurality of sets (2b and 2c, 2d and 2e, 2f and 2g, 2h and 2i, 2j and 2k, 2m and 2n) located in the central portion are connected only by the first pattern connection portions 3 (3a, 3b) and the third pattern connection portions 5 (5a, 5b). Thus, a ratio of each of the first pattern connection portions 3 (3a, 3b) and the third pattern connection portions 5 (5a, 5b) is 50% between the annular bodies of the sets. As in this embodiment, it is preferable that the adjacent annular bodies 2 of a plurality of sets located in the central portion are connected by connection portions of different patterns.

In the stent 1a of this embodiment, the first pattern connection portions 3 (3a, 3b) and the third pattern connection portions 5 (5a, 5b) are alternately arranged in the circumferential direction and inclined in the same direction.

In addition, the first pattern connection portion 3a and the first pattern connection portion 3b adjacent to each other in the axial direction of the stent 1a are inclined in different circumferential directions, and similarly, the third pattern connection portion 5a and the third pattern connection portion 5b adjacent to each other in the axial direction of the stent 1a are also inclined in different circumferential directions. Furthermore, in the stent 1a of this embodiment, the first pattern connection portion 3 (3a, 3b) and the third pattern connection portion 5 (5a, 5b) have substantially the same axial length.

In addition, in the stent 1a of this embodiment, as illustrated in FIGS. 3 and 4, the annular bodies 2 (2a and 2b, 2c and 2d, 2e and 2f, 2g and 2h, 2i and 2j, 2k and 2m, 2n and 2p) of a plurality of sets (second sets) other than the adjacent annular bodies 2 (2b and 2c, 2d and 2e, 2f and 2g, 2h and 2i, 2j and 2k, 2m and 2n) of a plurality of sets (first sets) located in the central portion are all connected by short fourth pattern connection portions 6 (6a, 6b) that connect the one end side apexes 25 (for example, 25f and 25h) and the other end side apexes 26 (for example, 26e, 26g).

The connection portions connected by the apexes and the apexes have a relatively stronger resistance to axial compression, are rather easy to release the stent, and are less likely to cause shortening. In addition, mounting is facilitated as a manufacturing advantage.

Further, in the stent 1a of this embodiment, the fourth pattern connection portions 6 (6a, 6b) adjacent to each other in the circumferential direction of the stent are inclined in the same circumferential direction, and the fourth pattern connection portion 6a and the fourth pattern connection portion 6b adjacent to each other in the axial direction of the stent 1a are inclined in different circumferential directions. The fourth pattern connection portion 6a and the fourth pattern connection portion 6b have substantially the same axial length.

In the stent 1a in this embodiment, the fourth pattern connection portion 6 (6a, 6b) has a shorter axial length than the first pattern connection portion 3 (3a, 3b) and the third pattern connection portion 5 (5a, 5b). Thus, a distance between the annular bodies 2 (between 2a and 2b, between 2c and 2d, between 2e and 2f, between 2g and 2h, between 2i and 2j, between 2k and 2m, between 2n and 2p) of a second set is shorter than a distance between the annular bodies 2 (between 2b and 2c, between 2d and 2e, between 2f and 2g, between 2h and 2i, between 2j and 2k, between 2m and 2n) of a first set.

Also in the stent 1a of this embodiment, substantially rectangular closed cells inclined in the first circumferential direction (first direction) of the stent are arranged in parallel in the circumferential direction. Further, substantially rectangular closed cells inclined in the second direction opposite to the first circumferential direction of the stent 1a are arranged in parallel in the circumferential direction adjacent in the axial direction to the substantially rectangular closed cells. Furthermore, in the stent 1a of this embodiment, one end portions of the substantially rectangular closed cells inclined in the second direction enter recesses between the other ends of the substantially rectangular closed cells inclined in the first direction. Thus, expansion holding force can be provided.

The stent 1a of this embodiment includes one end portion annular body connection portions that connect the other end side bent portions of the annular bodies located at one end and the one end side bent portions of the annular bodies adjacent to the annular bodies located at one end, and other end portion annular body connection portions that connect the one end side bent portions of the annular bodies located at the other end and the other end side bent portions of the annular bodies adjacent to the annular bodies located at the other end. The one end portion annular body connection portions include the fourth pattern connection portions 6, and the other end portion annular body connection portions include the fourth pattern connection portions 6.

Specifically, in the stent 1a of this embodiment, as illustrated in FIG. 3, the other end side apex 26 of the annular body 2a located at one end and the one end side apex 25 of the annular body 2b adjacent to the annular body 2a located at one end are connected by the fourth pattern connection portion 6a. The one end side apex 25 of the annular body 2p located at the other end and the other end side apex 26 of the annular body 2n adjacent to the annular body 2p located at the other end are connected by the fourth pattern connection portion 6a. The connection portion 6a has a shorter axial length of the stent than the connection portion 3 and the connection portion 5. Thus, in the stent 1a of this embodiment, so-called closed cells having a substantially rhombic shape are provided at both ends, so that the stent 1a has relatively high axial rigidity.

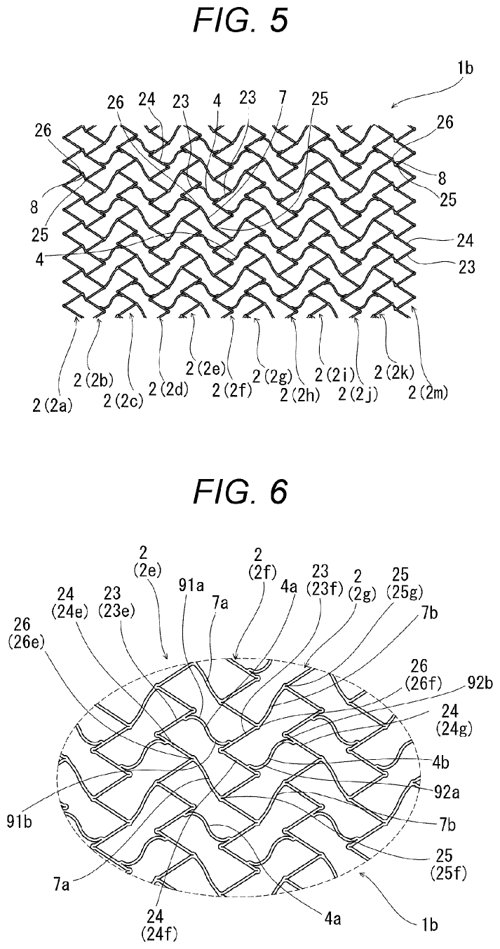

Next, a stent 1b of the embodiment illustrated in FIGS. 5 and 6 will be described.

In the stent 1b of this embodiment, as illustrated in FIGS. 5 and 6, the adjacent annular bodies 2 (2b, 2c, 2d, 2e, 2f, 2g, 2h, 2i, 2j, 2k) of a plurality of sets located in the central portion are connected by the second pattern connection portions 4 (4a, 4b) that connect the connection linear portions 23, 24 (for example, 23e, 24f) and the connection linear portions 23, 24 (for example, 23e, 24f) located closer to the other end side than the connection linear portions 23, 24 (for example, 23f, 24g), and long fourth pattern connection portions 7 (7a, 7b) that connect the one end side apexes 25 (for example, 25f, 25g) and the other end side apexes 26 (for example, 26e, 26f) located closer to the one end side than the one end side apexes 25 (for example, 25f, 25g).

The second pattern connection portion 4 (4a, 4b) connects the connection linear portions, and thus, when the second pattern connection portion 4 (4a, 4b) is disposed at the bent portion of the blood vessel, axial compressive force to be applied from the bent portion can be changed to act in the rotational direction, so that buckling can be suppressed, and favorable blood vessel followability can be provided.

The long fourth pattern connection portion 7 (7a, 7b) connects the adjacent apexes, and thus, when a diameter of the stent is reduced, rigidity in the axial direction can be improved by sandwiching the connection portion between the apexes, so that it is relatively easy to store the stent in the sheath, it is relatively easy to release the stent from the sheath, and it is difficult to shorten the axial length of the stent at the time of expansion.

In the stent 1b of this embodiment, adjacent annular bodies 2 of a plurality of sets are connected only by the second pattern connection portions 4 (4a, 4b) and the fourth pattern connection portions 7 (7a, 7b).

Furthermore, the second pattern connection portions 4 (4a, 4b) and the fourth pattern connection portions 7 (7a, 7b) are alternately arranged in the circumferential direction of the stent and are inclined in the same circumferential direction. In addition, the second pattern connection portion 4a and the second pattern connection portion 4b adjacent to each other in the axial direction of the stent 1b are inclined in different directions, and similarly, the fourth pattern connection portion 7a and the fourth pattern connection portion 7b adjacent to each other in the axial direction of the stent 1b are also inclined in different circumferential directions. Furthermore, in the stent 1b of this embodiment, the second pattern connection portion 4 (4a, 4b) has a longer axial length than the fourth pattern connection portion 7 (7a, 7b).

Furthermore, in the stent 1b of this embodiment, one end of the second pattern connection portion 4 (4a, 4b) is connected to the other end side bent portion side of the connection linear portion 23 or 24 (for example, 23e and 24f). Specifically, one end of the second pattern connection portion 4 (4a, 4b) is connected to a position closer to the other end side apex than the central portion of the connection linear portion 23 or 24 (for example, 23e, 24f). The other end of the second pattern connection portion 4 (4a, 4b) is connected to the one end side bent portion side of the connection linear portion 23 or 24 (for example, 23f, 24g). Specifically, the other end of the second pattern connection portion 4 (4a, 4b) is connected to a position closer to the one end side apex than the central portion of the connection linear portion 23 or 24 (for example, 23f, 24g).

In addition, in the stent 1b of this embodiment, the second pattern connection portion 4a includes a one end curved portion 91a curved in the direction opposite to the inclination direction of the connection portion (upward at the right-downward connection portion in FIG. 6) and an other end curved portion 91b curved in the inclination direction of the connection portion (downward at the right-downward connection portion in FIG. 6). Similarly, the second pattern connection portion 4b includes a one end curved portion 92a curved in a direction opposite to the inclination direction of the connection portion (downward at the right-upward connection portion in FIG. 6) and an other end curved portion 92b curved in the inclination direction of the connection portion (upward at the right-upward connection portion in FIG. 6). The apex of each curved portion is close to the one end side apex 25 or the other end side apex 26 of the connection linear portion to which the connection portion 4 (4a, 4b) is connected.

The stent 1b of this embodiment has a plurality of convex portions extending on an outer surface of the stent 1b. Specifically, the second pattern connection portion 4a and the second pattern connection portion 4b protrude outward at the time of expansion of the stent to form the convex portions with a protrusion amount increasing from both ends of the connection portion toward the central portion, on the outer surface of the stent, and the entire second pattern connection portion 4a and the second pattern connection portion 4b form the convex portions. The central portion of the convex portions formed by the entire second pattern connection portion 4a and the entire second pattern connection portion 4b protrudes most. A height of the convex portions can be, for example, preferably equal to or greater than 50 μm.

In the stent 1b of this embodiment, substantially rectangular closed cells inclined in the first circumferential direction (first direction) of the stent are arranged in parallel in the circumferential direction. Further, substantially rectangular closed cells inclined in the second direction opposite to the first circumferential direction of the stent 1b are arranged in parallel in the circumferential direction adjacent in the axial direction to the substantially rectangular closed cells. Furthermore, in the stent 1b of this embodiment, one end portions of the substantially rectangular closed cells inclined in the second direction enter recesses between the other ends of the substantially rectangular closed cells inclined in the first direction. Thus, expansion holding force can be provided.

As illustrated in FIG. 5, the stent 1b of this embodiment includes: the one end portion annular body connection portion 8 that connects the other end side apex 26 of the annular body 2a located at one end and the one end side apex 25 of the annular body 2b adjacent to the annular body 2a located at one end; and the other end portion annular body connection portion 8 that connects the one end side apex 25 of the annular body 2m located at the other end and the other end side apex 26 of the annular body 2k adjacent to the annular body 2m located at the other end. The connection portion 8 has a shorter axial length of the stent than the second pattern connection portion 4 (4a, 4b) and the fourth pattern connection portion 7 (7a, 7b). Thus, in the stent 1b of this embodiment, so-called closed cells having a substantially rhombic shape are provided at both ends, so that the stent 1b has relatively high axial rigidity.

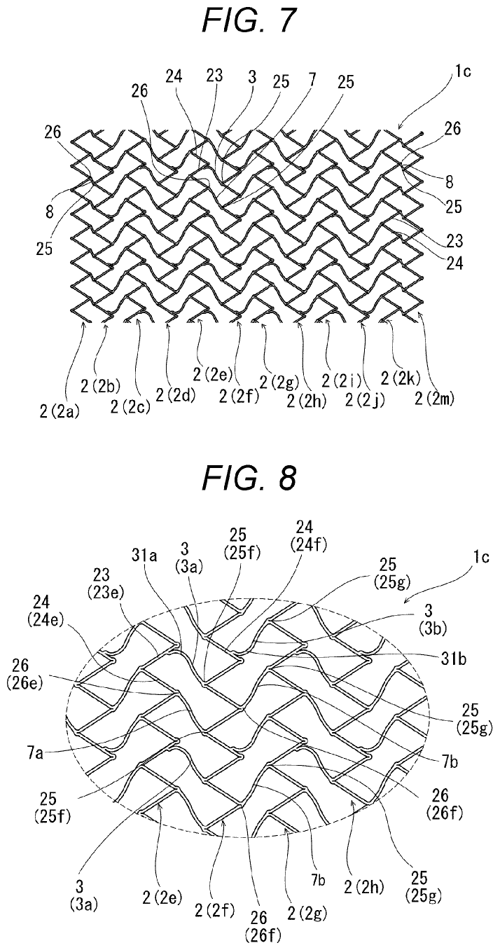

Next, a stent 1c of the embodiment illustrated in FIGS. 7 and 8 will be described.

In the stent 1c of this embodiment, as illustrated in FIGS. 7 and 8, between the adjacent annular bodies 2 (2b, 2c, 2d, 2e, 2f, 2g, 2h, 2i, 2j, 2k) of a plurality of sets located in the central portion are connected by the first pattern connection portions 3 (3a, 3b) that connect the one end side apexes 25 (for example, 25f, 25g) and the connection linear portions 23 or 24 (for example, 23e, 24f) located closer to one end side than the one end side apexes 25 (for example, 25f, 25g) as a basic pattern and further by long fourth pattern connection portions 7 (7a, 7b) that connect the other end side apexes 26 (for example, 26e, 26f) and the one end side apexes 25 (for example, 25f, 25g) located closer to the other end side than the other end side apexes 26 (for example, 26e, 26f).

In the stent of the present disclosure, the number of connection portions that connect adjacent annular bodies located in the central portion is preferably the same as, larger by one than, or smaller by one than the number of the one end side apexes or the number of the other end side apexes of the annular bodies connected by the connection portions.

In the stent 1c of this embodiment, all the adjacent annular bodies located in the central portion are connected by the first pattern connection portions 3 (3a, 3b) or the fourth pattern connection portions 7 (7a, 7b), and the first pattern connection portions 3 and the fourth pattern connection portions 7 are alternately arranged in the circumferential direction between the adjacent annular bodies.

In the stent 1c of this embodiment, the adjacent annular bodies 2 (2b, 2c, 2d, 2e, 2f, 2g, 2h, 2i, 2j, 2k) of a plurality of sets located in the central portion are connected only by the first pattern connection portions 3 (3a, 3b) and the fourth pattern connection portions 7 (7a, 7b). Thus, a ratio of each of the first pattern connection portions 3 (3a, 3b) and the fourth pattern connection portions 7 (7a, 7b) can be, for example, 50%.

Then, in the stent 1c of this embodiment, the first pattern connection portions 3 (3a, 3b) and the fourth pattern connection portions 7 (7a, 7b) are alternately arranged in the circumferential direction of the stent and are inclined in the same direction.

In addition, the first pattern connection portion 3a and the first pattern connection portion 3b adjacent to each other in the axial direction of the stent 1c are inclined in different circumferential directions, and similarly, the fourth pattern connection portion 7a and the fourth pattern connection portion 7b adjacent to each other in the axial direction of the stent 1c are also inclined in different circumferential directions. Furthermore, in the stent 1c of this embodiment, the first pattern connection portion 3 (3a, 3b) has a longer axial length than the fourth pattern connection portion 7 (7a, 7b).

Furthermore, in the stent 1c of this embodiment, one end of the first pattern connection portion 3 (3a, 3b) is connected to the other end side bent portion side of the connection linear portion 23 or 24 (for example, 23e, 24f). Specifically, one end of the first pattern connection portion 3 (3a, 3b) is connected to a position closer to the other end side apex than the central portion of the connection linear portion 23 or 24 (for example, 23e, 24f).

In addition, in the stent 1c of this embodiment, the first pattern connection portion 3a includes a one end curved portion 31a curved in a direction opposite to the inclination direction of the connection portion 3a (upward at the right-downward connection portion in FIG. 8). An apex of the curved portion 31a is close to the other end side apex 26 of the connection linear portion to which the connection portion 3a is connected. The first pattern connection portion 3b includes a one end curved portion 31b curved in a direction opposite to the inclination direction of the connection portion (downward at the right-upward connection portion in FIG. 8). An apex of the curved portion 31b is close to the other end side apex 26 of the connection linear portion to which the connection portion 3b is connected.

In the stent 1c of this embodiment, the first pattern connection portion of the stent 1c has a plurality of convex portions extending to the outer surface side. Specifically, the first pattern connection portion 3 (3a, 3b) protrudes outward at the time of expansion of the stent to form the convex portions with a protrusion amount increasing from both ends of the connection portion toward the central portion on the outer surface of the stent. A height of the convex portions is preferably, for example, equal to or greater than 50 μm. A distance between the convex portions in the axial direction is substantially the same as a distance between the other end side apexes of the adjacent annular bodies.

In the stent 1c of this embodiment, substantially rectangular closed cells inclined in the first circumferential direction (first direction) of the stent are arranged in parallel in the circumferential direction. Further, substantially rectangular closed cells inclined in the second direction opposite to the first circumferential direction of the stent 1c are arranged in parallel in the circumferential direction adjacent in the axial direction to the substantially rectangular closed cells. Furthermore, in the stent 1c of this embodiment, one end portions of the substantially rectangular closed cells inclined in the second direction enter recesses between the other ends of the substantially rectangular closed cells inclined in the first direction. Thus, expansion holding force is provided.

Furthermore, as illustrated in FIG. 7, the stent 1c of this embodiment includes the one end portion annular body connection portion 8 that connects the other end side apex 26 of the annular body 2a located at one end and the one end side apex 25 of the annular body 2b adjacent to the annular body 2a located at one end, and the other end portion annular body connection portion 8 that connects the one end side apex 25 of the annular body 2m located at the other end and the other end side apex 26 of the annular body 2k adjacent to the annular body 2m located at the other end. The connection portion 8 has a shorter axial length of the stent than the connection portion 3 and the connection portion 7. Thus, in the stent 1c of this embodiment, so-called closed cells having a substantially rhombic shape are provided at both ends, so that the stent 1c has relatively high axial rigidity.

Note that, as described above, the stent 1c of the present embodiment does not have the other end side bent portion that becomes a free end except for the annular body 2m located at the other end of the stent 1c and can be restored in the sheath 12 again even if the stent is exposed from the sheath to a certain extent.

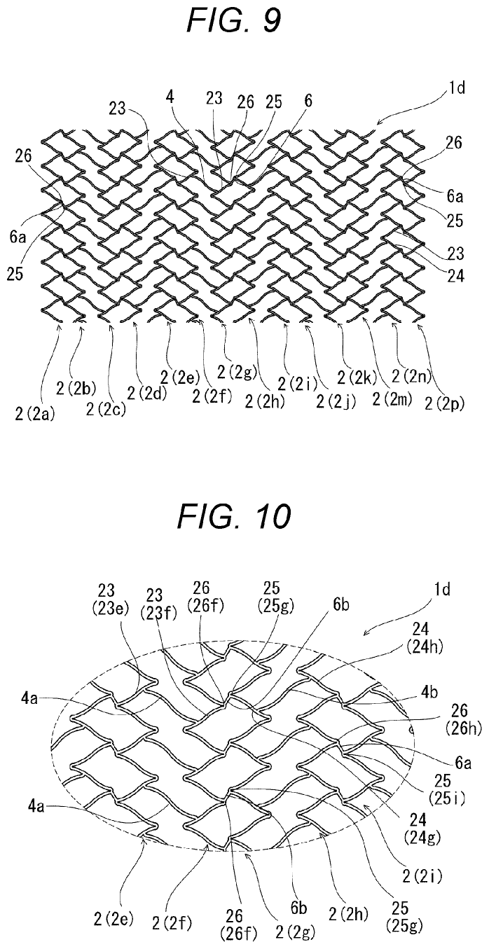

Next, a stent 1d of the embodiment illustrated in FIGS. 9 and 10 will be described.

In the stent 1d of this embodiment, as illustrated in FIGS. 9 and 10, the adjacent annular bodies 2 (2b and 2c, 2d and 2e, 2f and 2g, 2h and 2i, 2j and 2k, 2m and 2n) of a plurality of adjacent sets (first sets) located in the central portion are connected by the second pattern connection portions 4 (4a, 4b) that connect the connection linear portions 23, 24 (for example, 23e, 24g) and the connection linear portions 23, 24 (for example, 23e, 24g) located on the other end side of the connection linear portions 23, 24 (for example, 23f and 24h).

In the stent 1d of this embodiment, the adjacent annular bodies (2b and 2c, 2d and 2e, 2f and 2g, 2h and 2i, 2j and 2k, 2m and 2n) of a plurality of sets located in the central portion are connected only by the second pattern connection portions 4 (4a, 4b). In addition, the second pattern connection portion 4a and the second pattern connection portion 4b adjacent to each other in the axial direction of the stent 1d are inclined in different circumferential directions.

In addition, in the stent 1d of this embodiment, as illustrated in FIGS. 9 and 10, the annular bodies 2 (2a and 2b, 2c and 2d, 2e and 2f, 2g and 2h, 2i and 2j, 2k and 2m, 2n and 2p) of a plurality of sets (second sets) other than the adjacent annular bodies 2 (2b and 2c, 2d and 2e, 2f and 2g, 2h and 2i, 2j and 2k, 2m and 2n) of a plurality of sets (first sets) located in the central portion are all connected by short fourth pattern connection portions 6 (6a, 6b) that connect the one end side apexes 25 (for example, 25g, 25i) and the other end side apexes 26 (for example, 26f, 26h).

Thus, in a case where the first set and the second set are combined, the number of the second pattern connection portions 4 (4a, 4b) and the number of the fourth pattern connection portions 6 (6a, 6b) are the same, and a ratio of the second pattern connection portions 4 (4a, 4b) located in the central portion can be, for example, 50% of a total number of connection portions of the central portion.

In the stent 1d of this embodiment, the fourth pattern connection portions 6 (6a, 6b) adjacent to each other in the circumferential direction of the stent are inclined in the same direction, and the fourth pattern connection portions 6a and the fourth pattern connection portions 6b adjacent to each other in the axial direction of the stent 1d are inclined in different circumferential directions. The fourth pattern connection portion 6a and the fourth pattern connection portion 6b have substantially the same axial length.

In the stent 1d in this embodiment, the fourth pattern connection portion 6 (6a, 6b) has a shorter axial length than the second pattern connection portion 4 (4a, 4b). Thus, a distance between the annular bodies 2 (between 2a and 2b, between 2c and 2d, between 2e and 2f, between 2g and 2h, between 2i and 2j, between 2k and 2m, between 2n and 2p) of a second set is shorter than a distance between the annular bodies 2 (between 2b and 2c, between 2d and 2e, between 2f and 2g, between 2h and 2i, between 2j and 2k, between 2m and 2n) of a first set.

In the stent 1d of this embodiment, the second pattern connection portion of the stent 1d has a plurality of convex portions extending to the outer surface side. Specifically, the second pattern connection portion 4 (4a, 4b) protrudes outward at the time of expansion of the stent to form the convex portions with a protrusion amount increasing from both ends of the connection portion toward the central portion on the outer surface of the stent. A height of the convex portions is preferably, for example, equal to or greater than 50 μm. A distance between the convex portions in the axial direction is substantially the same as a distance between the other end side apexes of the adjacent annular bodies.

Also in the stent 1d of this embodiment, substantially rectangular closed cells inclined in the first circumferential direction (first direction) of the stent are arranged in parallel in the circumferential direction. Further, substantially rectangular closed cells inclined in the second direction opposite to the first circumferential direction of the stent 1d are arranged in parallel in the circumferential direction adjacent in the axial direction to the substantially rectangular closed cells. Furthermore, in the stent 1d of this embodiment, one end portions of the substantially rectangular closed cells inclined in the second direction enter recesses between the other ends of the substantially rectangular closed cells inclined in the first direction. Thus, expansion holding force is provided.

Furthermore, in the stent 1d of this embodiment, as illustrated in FIG. 9, the other end side apex 26 of the annular body 2a located at one end and the one end side apex 25 of the annular body 2b adjacent to the annular body 2a located at one end are connected by the connection portion 6a, and the one end side apex 25 of the annular body 2p located at the other end and the other end side apex 26 of the annular body 2n adjacent to the annular body 2p located at the other end are connected by the connection portion 6a. The connection portion 6a has a shorter axial length of the stent than the connection portion 4. Thus, the stent 1d of this embodiment is provided with so-called closed cells having a substantially rhombic shape at both ends, so that the stent 1d has relatively high axial rigidity.

As a result of comparing and evaluating the followability at the bent portion of the embodiments of the present disclosure, it has been found that the stent illustrated in FIGS. 1 and 2, for example, has the best followability, the stent illustrated in FIGS. 5 and 6, the stent illustrated in FIGS. 7 and 8, and the stent illustrated in FIGS. 9 and 10, for example, have the second best followability, and the stent illustrated in FIGS. 3 and 4, for example, has the third best followability.

In all the embodiments described above, the axial length of the first pattern connection portion 3 (3a, 3b) is preferably, for example, 0.2 mm to 3.0 mm, and particularly preferably 0.5 mm to 2.0 mm. In addition, an inclination angle of the first pattern connection portion 3 (3a, 3b) with respect to the central axis of the stent is preferably, for example, 40 degrees to 70 degrees, and more preferably 45 degrees to 65 degrees.

The axial length of the second pattern connection portion 4 (4a, 4b) is preferably, for example, 0.2 mm to 3.0 mm, and more preferably 0.5 mm to 2.0 mm. In addition, an inclination angle of the second pattern connection portion 4 (4a, 4b) with respect to the central axis of the stent is preferably, for example, 40 degrees to degrees, and more preferably 45 degrees to 65 degrees.

The axial length of the third pattern connection portion 5 (5a, 5b) is preferably, for example, 0.2 mm to 3.0 mm, and more preferably 0.5 mm to 2.0 mm. In addition, an inclination angle of the third pattern connection portion 5 (5a, 5b) with respect to the central axis of the stent is preferably, for example, 40 degrees to 70 degrees, and more preferably 45 degrees to 65 degrees.

The axial length of the fourth pattern connection portion 6 (6a, 6b) is preferably, for example, 0.1 mm to 1.5 mm, and more preferably 0.15 mm to 1.0 mm. In addition, an inclination angle of the fourth pattern connection portion 6 (6a, 6b) with respect to the central axis of the stent is preferably, for example, 40 degrees to degrees, and more preferably 45 degrees to 65 degrees.

The axial length of the fourth pattern connection portion 7 (7a, 7b) is preferably, for example, 0.2 mm to 3.0 mm, and more preferably 0.5 mm to 2.0 mm. In addition, an inclination angle of the fourth pattern connection portion 7 (7a, 7b) with respect to the central axis of the stent is preferably, for example, 40 degrees to 70 degrees, and more preferably 45 degrees to 65 degrees.

A superelastic metal is suitable as a constituent material of the stent. As the superelastic metal, a superelastic alloy is suitably used. The superelastic alloy herein is generally called a shape memory alloy and exhibits superelasticity at least at a biological temperature (around 37° C.). Particularly preferably, a superelastic alloy such as a Ti—Ni alloy including Ni of 49 atomic % to 53 atomic %, a Cu—Zn alloy including Zn of 38.5 weight % to 41.5 weight %, a Cu—Zn—X alloy (X=Be, Si, Sn, Al, Ga) including X of 1 weight % to 10 weight %, a Ni—Al alloy including Al of 36 atomic % to 38 atomic %, or a Mg—Sc alloy including Sc of 15 atomic % to 25 atomic % is suitably used. The Ti—Ni alloy is particularly preferable. In addition, mechanical characteristics can be appropriately changed by using a Ti—Ni—X alloy (X=Co, Fe, Mn, Cr, V, Al, Nb, W, B, etc.) in which a part of the Ti—Ni alloy is substituted with 0.01 weight % X to 10.0 weight % X, using a Ti—Ni—Y alloy (Y=Cu, Pb, Zr) in which a part of the Ti—Ni alloy is substituted with 0.01 weight % Y to 30.0 weight % Y, and selecting a cold working ratio or/and conditions of a final heat treatment.

In addition, the mechanical characteristics can be appropriately changed by using the Ti—Ni alloy, the Ti—Ni—X alloy, or the Ti—Ni—Y alloy and selecting the cold working ratio and/or the conditions of the final heat treatment. Buckling strength of the superelastic alloy to be used (yield stress at the time of loading at the time of developing superelastic characteristics) is 5 kgf/mm² to 200 kgf/mm² (22° C.), more preferably 8 kgf/mm² to 150 kgf/mm², and a restoring stress (yield stress at the time of unloading) is 3 kgf/mm² to 180 kgf/mm² (22° C.), more preferably 5 kgf/mm² to 130 kgf/mm². Superelasticity as used herein means that even if a normal metal is deformed (bent, pulled, compressed) to a region where the metal is plastically deformed at an operating temperature, the metal is restored to a substantially pre-compressed shape without requiring heating after the deformation is released.

Then, as the stent 1, for example, in a case where the stent is used for expansion of a cerebral blood vessel, the diameter at the time of expansion (at the time of non-compression) is preferably, for example, about 0.5 mm to 6.0 mm, and more preferably 0.9 mm to 5.0 mm. In addition, a length of the stent at the time of expansion (at the time of non-compression) is preferably, for example, about 5 mm to 50 mm.

A thickness of the stent is preferably, for example, about 0.05 mm to 0.15 mm, and more preferably 0.06 mm to 0.13 mm. A width of a linear component constituting the stent is preferably, for example, about 0.04 mm to 0.15 mm, and more preferably 0.05 mm to 0.13 mm.

In addition, in the stent of the present disclosure, the stent may contain a physiologically active substance so as to be releasable. As a method of containing a physiologically active substance so as to be releasable, for example, there is a method of coating a surface of the stent with a polymer (for example, a biodegradable polymer) containing a physiologically active substance.

The biodegradable polymer is not particularly limited as long as it is enzymatically or non-enzymatically decomposed in vivo and the decomposed product does not exhibit toxicity, and for example, polylactic acid, polyglycolic acid, a polylactic acid-polyglycolic acid copolymer, polycaprolactone, a polylactic acid-polycaprolactone copolymer, a polyorthoester, polyphosphazene, a polyphosphoric acid ester, polyhydroxybutyric acid, polymalic acid, poly-a-amino acid, collagen, gelatin, laminin, heparan sulfate, fibronectin, vitronectin, chondroitin sulfate, hyaluronic acid, polypeptide, chitin, chitosan, and the like, can be used.

In addition, as the physiologically active substance, a substance that promotes melting or metabolism of thrombus or thrombus complex, a substance that suppresses increase in thrombus or thrombus complex, a substance that suppresses intimal thickening, an anticancer drug, an immunosuppressive agent, an antibiotic, an antirheumatic agent, an antithrombotic agent, an HMG-CoA reductase inhibitor, an ACE inhibitor, a calcium antagonist, an antilipemia agent, an anti-inflammatory agent, an integrin inhibitor, an antiallergic agent, an antioxidant, a GPIIbIIIa antagonist, a retinoid, a flavonoid and a carotenoid, a lipid improver, a DNA synthesis inhibitor, a tyrosine kinase inhibitor, an antiplatelet agent, a vascular smooth muscle proliferation inhibitor, a biological material, interferon, and an epithelial cell generated by genetic engineering are used. Further, a mixture of two or more kinds of the above-mentioned substances, and the like, may be used.

As the substance that promotes melting or metabolism of thrombus or thrombus complex or the substance that suppresses increase in thrombus or thrombus complex, streptokinase, plasminogen activator, urokinase, stafinokinase, lumbrokinase, nattokinase, or an analog thereof can be used. In addition, as the substance that suppresses increase in thrombus or a thrombus complex, an antiplatelet drug represented by acetylsalicylic acid, ticlopidine, dipyridamole, cilostazol, beraprost Na, rimaprost alfathecus, ethyl icosapentoenate, salvogrelate hydrochloride, trapidyl, clopidogrel, prasugrel, and analogs thereof, or an anticoagulant represented by a GP IIb/IIIa antagonist, heparin, or warfarin potassium can be used.

Next, a stent delivery system of an embodiment of the present disclosure will be described using an embodiment illustrated in the drawings.

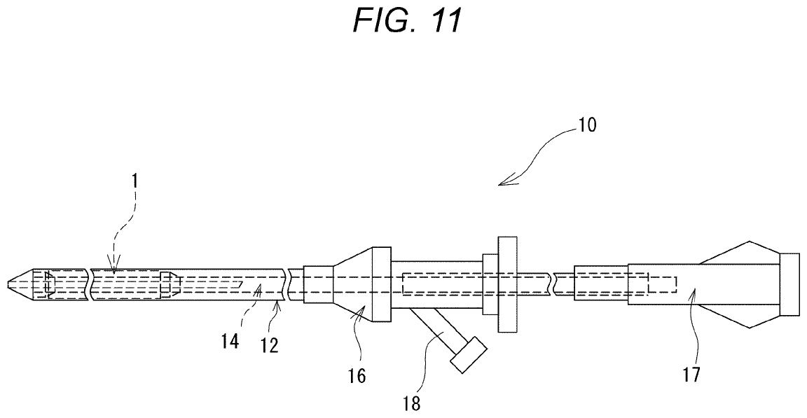

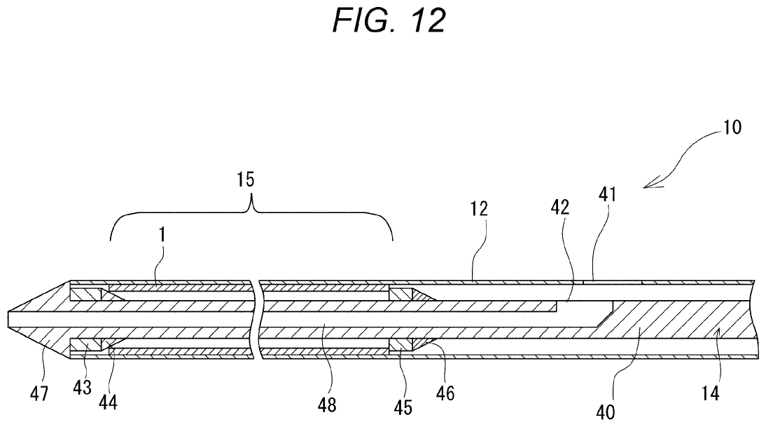

FIG. 11 is a partially omitted front view of the stent delivery system of the embodiment of the present disclosure. FIG. 12 is an enlarged longitudinal cross-sectional view of the vicinity of a distal end portion of the stent delivery system illustrated in FIG. 11.

A stent delivery system 10 of this embodiment includes the sheath 12, the stent 1 stored in a distal end portion of the sheath 12, and the inner tube 14 that is to be slidably inserted through the sheath 12 and releases the stent 1 from the distal end of the sheath 12.

In the stent delivery system 10 of this embodiment, as the stent 1, the above-described self-expanding stent is used which is formed in a cylindrical shape, is compressed in the central axis direction at the time of in-vivo insertion, and expands outward so as to be able to restore the shape before compression at the time of in-vivo indwelling.

As illustrated in FIG. 11, the stent delivery system 10 of this embodiment includes the sheath 12, the self-expanding stent 1, and the inner tube 14.

As illustrated in FIGS. 11 and 12, the sheath 12 is a tubular body and has openings at the distal end and a proximal end of the tubular body. The opening at the distal end of the tubular body functions as a release port of the stent 1 when the stent 1 is indwelled in a stenosed part in a body cavity. By sliding the sheath 12 toward the proximal end side, the stent 1 is released from the opening at the distal end of the tubular body, stress load is released, and the stent 1 expands and returns to the shape before compression. The distal end portion of the sheath 12 is a stent storage portion 15 that stores the stent 1 in the stent storage portion 15. In addition, the sheath 12 includes a side hole 41 provided on a proximal end side of the storage portion 15. The side hole 41 is provided for leading a guide wire to the outside.

An outer diameter of the sheath 12 is preferably, for example, about 0.4 mm to 4.0 mm, and more preferably 0.5 mm to 3.0 mm. An inner diameter of the sheath 12 is preferably, for example, about 0.3 mm to 2.0 mm. A length of the sheath 12 is preferably, for example, about 300 mm to 2500 mm, more about 300 mm to 2000 mm.

As illustrated in FIG. 11, a sheath hub 16 is fixed to a proximal end portion of the sheath 12. The sheath hub 16 can include a sheath hub body and a valve body that is stored in the sheath hub body and slidably and liquid-tightly holds the inner tube 14. In addition, the sheath hub 16 can include a side port 18 branching obliquely rearward from the vicinity of the center of the sheath hub body. The sheath hub 16 preferably includes an inner tube lock mechanism that restricts movement of the inner tube 14.

As illustrated in FIGS. 11 and 12, the inner tube 14 includes a shaft-like inner tube main body 40, a distal end portion 47 provided at a distal end of the inner tube main body 40 and protruding from the distal end of the sheath 12, and an inner tube hub 17 fixed to a proximal end portion of the inner tube main body 40.

The distal end portion 47 is preferably formed in a tapered shape protruding from the distal end of the sheath 12 and gradually reducing in diameter toward the distal end as illustrated in FIG. 12. This configuration facilitates insertion into the stenosed part. In addition, it is preferable that the inner tube 14 is provided on the distal end side of the stent 1 and includes a stopper that can inhibit movement of the sheath in a distal end direction. A proximal end of the distal end portion 47 can abut against the distal end of the sheath 12 and functions as the above-described stopper.

In addition, as illustrated in FIG. 12, the inner tube 14 can include two protrusions 43 and 45 for holding the self-expanding stent 1. The protrusions 43 and are preferably circular protrusions. A stent holding protrusion 43 is provided on the proximal end side of the distal end portion 47 of the inner tube 14. A stent extrusion protrusion 45 is provided on the proximal end side of the stent holding protrusion 43 by a predetermined distance. The stent 1 is disposed between the two protrusions 43 and 45. The protrusions 43 and 45 have an outer diameter so that the protrusions 43 and 45 can abut against the compressed stent 1 described later.

Thus, movement of the stent 1 to the distal end side is restricted by the protrusion 43, and movement to the proximal end side is restricted by the protrusion 45. Furthermore, when the inner tube 14 moves to the distal end side, the stent 1 is pushed to the distal end side by the protrusion 45 and discharged from the sheath 12. Furthermore, as illustrated in FIG. 12, the proximal end side of the stent extrusion protrusion 45 preferably has a tapered portion 46 whose diameter gradually decreases toward the proximal end side. Similarly, as illustrated in FIG. 12, the proximal end side of the stent holding protrusion 43 preferably has a tapered portion 44 whose diameter gradually decreases toward the proximal end side.

In this manner, when the inner tube 14 protrudes from the distal end of the sheath 12 and the inner tube 14 is restored in the sheath 12 after the stent 1 is released from the sheath, the protrusion is prevented from being caught by the distal end of the sheath. The protrusions 43 and 45 may be formed with different members using X-ray contrast materials. As a result, a position of the stent can be accurately grasped under X-ray imaging, which facilitates procedure.

As illustrated in FIG. 12, the inner tube 14 can include a lumen 48 extending at least from the distal end to the proximal end side of the stent storage portion 15 of the sheath 12 and an inner tube side hole 42 communicating with the lumen 48 on the proximal end side of the stent storage portion. In the stent delivery system 10 of this embodiment, the lumen 48 terminates at a portion where the side hole 42 is formed. The lumen 48 is provided for inserting one end of the guide wire from the distal end of the stent delivery system 10, partially inserting the guide wire through the inner tube, and then leading the guide wire to the outside from a side surface of the inner tube. The side hole 42 of the inner tube is located slightly closer to the distal end side of the stent delivery system 10 from the side hole 41 of the sheath. The center of the side hole 42 of the inner tube is preferably, for example, 0.5 mm to 10 mm distal from the center of the side hole 41 of the sheath.

Note that the stent delivery system is not limited to the above-described type, and the lumen 48 may extend to the proximal end of the inner tube. In this case, the side hole 41 of the sheath is unnecessary.

The inner tube 14 penetrates inside of the sheath 12 and protrudes from the opening at the proximal end of the sheath 12. As illustrated in FIG. 11, an inner tube hub 17 is fixed to a proximal end portion of the inner tube 14.

In addition, in the stents of all the above-described embodiments, the stent may include contrast markers at one end portion and the other end portion. The contrast marker may be any marker for X-ray contrast, ultrasound contrast, or the like. The marker is formed with a contrast substance such as an X-ray contrast substance or an ultrasonic contrast substance. As a material for forming the marker, for example, gold, platinum, tungsten, iridium, palladium, or an alloy thereof, or a gold-palladium alloy, platinum-iridium, NiTiPd, NiTiAu, or the like, is suitable.

The in-vivo indwelling stent of the present disclosure is an in-vivo indwelling stent in which a plurality of annular bodies formed in a circular shape with linear components are arranged in the axial direction, and adjacent annular bodies are connected by connection portions. Each of the annular bodies is formed with one endless linear component having a plurality of one end side apexes located on one end side in the axial direction of the stent, a plurality of other end side apexes located on the other end side in the axial direction of the stent, and a plurality of connection linear portions that connect the one end side apexes and the other end side apexes. The adjacent annular bodies of a plurality of sets located at least in the central portion of the stent are connected by a plurality of connection portions, and, for example, 25% to 75% of a total number of the connection portions located in a central portion are basic pattern connection portions including first pattern connection portions that connect one end side apexes and connection linear portions located closer to one end side than the one end side apexes or second pattern connection portions that connect the connection linear portions and the connection linear portions located closer to the other end side than the connection linear portions, and remaining connection portions located in the central portion are pattern connection portions of at least one type selected from the first pattern connection portions or the second pattern connection portions which are not selected in the basic pattern connection portions, third pattern connection portions that connect the other end side apexes and the connection linear portions located closer to the other end side than the other end side apexes, and fourth pattern connection portions that connect the one end side apexes and the other end side apexes located closer to one end side than the one end side apexes, and the number of connection portions that connect the adjacent annular bodies located in the central portion is the same as, larger by one than, or smaller by one than the number of the one end side apexes or the number of the other end side apexes of the annular bodies connected by the connection portions.

Thus, favorable storability and followability by radial compression, and sufficient expansion force can be provided.

The in-vivo indwelling stent of the present invention is as follows.

(1) An in-vivo indwelling stent in which a plurality of annular bodies formed in a circular shape with linear components are arranged in an axial direction, and adjacent annular bodies are connected by connection portions, each of the annular bodies being formed with one endless linear component having a plurality of one end side apexes located on one end side in the axial direction of the stent, a plurality of other end side apexes located on the other end side in the axial direction of the stent, and a plurality of connection linear portions that connect the one end side apexes and the other end side apexes, and adjacent annular bodies of a plurality of sets located in at least a central portion of the stent being connected by a plurality of connection portions, and 25% to 75% of a total number of the connection portions located in the central portion are basic pattern connection portions including first pattern connection portions that connect the one end side apexes and the connection linear portions located closer to one end side than the one end side apexes, or second pattern connection portions that connect the connection linear portions and the connection linear portions located closer to the other end side than the connection linear portions, and remaining connection portions located in the central portion being connection portions of at least one type selected from the first pattern connection portions or the second pattern connection portions which are not selected in the basic pattern connection portions, third pattern connection portions that connect the other end side apexes and the connection linear portions located closer to the other end side apexes, fourth pattern connection portions that connect the one end side apexes and the other end side apexes located closer to one end side than the one end side apexes, and the number of connection portions that connect the adjacent annular bodies located in the central portion being the same as, larger by one than, or smaller by one than the number of the one end side apexes or the number of the other end side apexes of the annular bodies connected by the connection portions.

This in-vivo indwelling stent is an in-vivo indwelling stent in which a plurality of annular bodies formed in a circular shape with linear components are arranged in the axial direction, and adjacent annular bodies are connected by connection portions. Each of the annular bodies is formed with one endless linear component having a plurality of one end side apexes located on one end side in the axial direction of the stent, a plurality of other end side apexes located on the other end side in the axial direction of the stent, and a plurality of connection linear portions that connect the one end side apexes and the other end side apexes. The adjacent annular bodies of a plurality of sets located at least in the central portion of the stent are connected by a plurality of connection portions, and 25% to 75% of a total number of the connection portions located in a central portion are basic pattern connection portions including first pattern connection portions that connect one end side apexes and connection linear portions located closer to one end side than the one end side apexes or second pattern connection portions that connect the connection linear portions and the connection linear portions located closer to the other end side than the connection linear portions, and remaining connection portions located in the central portion are pattern connection portions of at least one type selected from the first pattern connection portions or the second pattern connection portions which are not selected in the basic pattern connection portions, third pattern connection portions that connect the other end side apexes and the connection linear portions located closer to the other end side than the other end side apexes, and fourth pattern connection portions that connect the one end side apexes and the other end side apexes located closer to one end side than the one end side apexes, and the number of connection portions that connect the adjacent annular bodies located in the central portion is the same as, larger by one than, or smaller by one than the number of the one end side apexes or the number of the other end side apexes of the annular bodies connected by the connection portions.

Thus, favorable storability and followability by radial compression, and sufficient expansion force are provided.

Furthermore, the above embodiment may be as follows.

(2) The in-vivo indwelling stent according to (1), in which the adjacent annular bodies of a plurality sets located in the central portion are connected by the first pattern connection portions that connect the one end side apexes and the connection linear portions located closer to one end side than the one end side apexes, and the third pattern connection portions that connect the other end side apexes and the connection linear portions located closer to the other end side than the other end side apexes, and the first pattern connection portions and the third pattern connection portions are alternately arranged in a circumferential direction between the adjacent annular bodies.

(3) The in-vivo indwelling stent according to (1), in which all the adjacent annular bodies located in the central portion are connected by the first pattern connection portions that connect the one end side apexes and the connection linear portions located closer to one end side than the one end side apexes, and the third pattern connection portions that connect the other end side apexes and the connection linear portions located closer to the other end side than the other end side apexes, and the first pattern connection portions and the third pattern connection portions are alternately arranged in the circumferential direction between the adjacent annular bodies.

(4) The in-vivo indwelling stent according to (1), in which all the adjacent annular bodies of a first set located in the central portion are connected by the second pattern connection portions that connect the connection linear portions and the connection linear portions located closer to the other end side than the connection linear portions, the stent includes a plurality of the first sets of annular bodies, and all of the adjacent first sets are connected by the fourth pattern connection portions that connect the one end side apexes and the other end side apexes.

(5) The in-vivo indwelling stent according to (1), in which, in the adjacent annular bodies located in the central portion, the second pattern connection portions that connect the connection linear portions and the connection linear portions located closer to the other end side than the connection linear portions and the fourth pattern connection portions that connect the one end side apexes and the other end side apexes are alternately arranged in a circumferential direction, the second pattern connection portions adjacent in the axial direction extend in different circumferential directions, and the fourth pattern connection portions adjacent in the axial direction extend in different circumferential directions.

(6) The in-vivo indwelling stent according to (1), in which the adjacent annular bodies of a first set located in the central portion are connected by the first pattern connection portions that connect the one end side apexes and the connection linear portions located closer to one end side than the one end side apexes, and the third pattern connection portions that connect the other end side apexes and the connection linear portions located closer to the other end side than the other end side apexes, and the first pattern connection portions and the third pattern connection portions are alternately arranged in a circumferential direction in the annular bodies of the first set.

(7) The in-vivo indwelling stent according to (1), in which the adjacent annular bodies of a first set located in the central portion are connected by the first pattern connection portions that connect the one end side apexes and the connection linear portions located closer to one end side than the one end side apexes, and the third pattern connection portions that connect the other end side apexes and the connection linear portions located closer to the other end side than the other end side apexes, and the first pattern connection portions and the third pattern connection portions are alternately arranged in a circumferential direction in the annular bodies of the first set, and the stent includes a plurality of the first sets of annular bodies, and the first sets adjacent to each other are all connected by the fourth pattern connection portions that connect the one end side apexes and the other end side apexes.

(8) The in-vivo indwelling stent according to (1), in which the adjacent annular bodies of a plurality of sets located in the central portion are connected by the first pattern connection portions that connect the one end side apexes and the connection linear portions located closer to one end side than the one end side apexes, and the fourth pattern connection portions that connect the one end side apexes and the other end side apexes, and the first pattern connection portions and the fourth pattern connection portions are alternately arranged in a circumferential direction.

(9) The in-vivo indwelling stent according to (1), in which all the adjacent annular bodies located in the central portion are connected by the first pattern connection portions that connect the one end side apexes and the connection linear portions located closer to one end side than the one end side apexes, and the fourth pattern connection portions that connect the one end side apexes and the other end side apexes located closer to one end side than the one end side apexes, and the first pattern connection portions and the fourth pattern connection portions are alternately arranged in a circumferential direction.

(10) The in-vivo indwelling stent according to any one of (1) to (9), in which the connection portions adjacent to each other in the axial direction of the stent in all the adjacent annular bodies located in the central portion extend in different circumferential directions.

(11) The in-vivo indwelling stent according to any one of (1) to (9), in which the connection portions of the same pattern adjacent to each other in the axial direction of the stent located in the central portion extend in different circumferential directions.

(12) The in-vivo indwelling stent according to any one of (4), (5), and (7) to (11), in which the stent includes: one end portion annular body connection portions that connect other end side bent portions having the other end side apexes of the annular body located at one end and one end side bent portions having the one end side apexes of the annular body adjacent to the annular body located at the one end; and other end portion annular body connection portions that connect one end side bent portions having the one end side apexes of the annular body located at the other end and other end side bent portions having the other end side apexes of the annular body adjacent to the annular body located at the other end, the one end portion annular body connection portions include the fourth pattern connection portions, and the other end portion annular body connection portions include the fourth pattern connection portions.

(13) The in-vivo indwelling stent according to any one of (1) to (12), in which the connection portions are inclined in the axial direction of the stent, and the connection portions that connect the adjacent annular bodies are all inclined in the same direction.

(14) The in-vivo indwelling stent according to any one of (1) to (13), including a plurality of convex portions on an outer surface of the stent.

(15) The in-vivo indwelling stent according to (14), in which a height of the convex portions is equal to or greater than 50 μm.

(16) The in-vivo indwelling stent according to any one of (1) to (15), in which the stent is formed in a substantially cylindrical shape, is compressed in a central axis direction at the time of in-vivo insertion and expands outward to restore a shape before compression at the time of in-vivo indwelling.

Further, the stent delivery system of the present invention is as follows.

(17) A stent delivery system including: a sheath; the stent according to (16) stored in a distal end portion of the sheath; and a shaft that is to be inserted into the sheath and releases the stent from a distal end of the sheath.

The detailed description above describes embodiments of an in-vivo indwelling stent and a stent delivery system to be used for improving stenosis or occlusion generated in a body lumen such as a blood vessel, a bile duct, a trachea, an esophagus, or a urethra. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An in-vivo indwelling stent comprising:
a plurality of annular bodies formed in a circular shape with linear components arranged in an axial direction, and adjacent annular bodies of the plurality of annual bodies connected by connection portions;
each of the plurality of annular bodies being formed with one endless linear component having a plurality of one end side apexes located on one end side in the axial direction of the stent, a plurality of other end side apexes located on the other end side in the axial direction of the stent, and a plurality of connection linear portions that connect the one end side apexes and the other end side apexes; and
the adjacent annular bodies including a plurality of sets located in at least a central portion of the stent being connected by a plurality of connection portions, and 25% to 75% of a total number of the plurality of connection portions located in the central portion being basic pattern connection portions including first pattern connection portions that connect the one end side apexes and the connection linear portions located closer to one end side than the one end side apexes or second pattern connection portions that connect the connection linear portions and the connection linear portions located closer to the other end side than the connection linear portions, and remaining connection portions of the plurality of connection portions located in the central portion being pattern connection portions of at least one type selected from the first pattern connection portions or the second pattern connection portions which are not selected in the basic pattern connection portions, third pattern connection portions that connect the other end side apexes and the connection linear portions located closer to the other end side than the other end side apexes, and fourth pattern connection portions that connect the one end side apexes and the other end side apexes located closer to one end side than the one end side apexes, and the number of connection portions that connect the adjacent annular bodies located in the central portion being the same as, larger by one than, or smaller by one than the number of the one end side apexes or the number of the other end side apexes of the annular bodies connected by the connection portions.

2. The in-vivo indwelling stent according to claim 1, wherein the adjacent annular bodies of the plurality of sets located in the central portion are connected by the first pattern connection portions that connect the one end side apexes and the connection linear portions located closer to one end side than the one end side apexes, and the third pattern connection portions that connect the other end side apexes and the connection linear portions located closer to the other end side than the other end side apexes, and the first pattern connection portions and the third pattern connection portions are alternately arranged in a circumferential direction between the adjacent annular bodies.

3. The in-vivo indwelling stent according to claim 1, wherein all the adjacent annular bodies located in the central portion are connected by the first pattern connection portions that connect the one end side apexes and the connection linear portions located closer to one end side than the one end side apexes, and the third pattern connection portions that connect the other end side apexes and the connection linear portions located closer to the other end side than the other end side apexes, and the first pattern connection portions and the third pattern connection portions are alternately arranged in a circumferential direction between the adjacent annular bodies.

4. The in-vivo indwelling stent according to claim 1, wherein all the adjacent annular bodies of a first set of the adjacent annular bodies located in the central portion are connected by the second pattern connection portions that connect the connection linear portions and the connection linear portions located closer to the other end side than the connection linear portions, the stent includes a plurality of the first sets of annular bodies, and all of the adjacent first sets of the adjacent annular bodies are connected by the fourth pattern connection portions that connect the one end side apexes and the other end side apexes.

5. The in-vivo indwelling stent according to claim 1, wherein in the adjacent annular bodies located in the central portion, the second pattern connection portions that connect the connection linear portions and the connection linear portions located closer to the other end side than the connection linear portions and the fourth pattern connection portions that connect the one end side apexes and the other end side apexes are alternately arranged in a circumferential direction, the second pattern connection portions adjacent in the axial direction extend in different circumferential directions, and the fourth pattern connection portions adjacent in the axial direction extend in different circumferential directions.

6. The in-vivo indwelling stent according to claim 1, wherein the adjacent annular bodies of a first set located in the central portion are connected by the first pattern connection portions that connect the one end side apexes and the connection linear portions located closer to one end side than the one end side apexes, and the third pattern connection portions that connect the other end side apexes and the connection linear portions located closer to the other end side than the other end side apexes, and the first pattern connection portions and the third pattern connection portions are alternately arranged in a circumferential direction in the annular bodies of the first set.

7. The in-vivo indwelling stent according to claim 1, wherein the adjacent annular bodies of a first set located in the central portion are connected by the first pattern connection portions that connect the one end side apexes and the connection linear portions located closer to one end side than the one end side apexes, and the third pattern connection portions that connect the other end side apexes and the connection linear portions located closer to the other end side than the other end side apexes, and the first pattern connection portions and the third pattern connection portions are alternately arranged in a circumferential direction in the annular bodies of the first set; and
the stent includes a plurality of the first sets of annular bodies, and the first sets adjacent to each other are all connected by the fourth pattern connection portions that connect the one end side apexes and the other end side apexes.

8. The in-vivo indwelling stent according to claim 1, wherein the adjacent annular bodies of the plurality of sets located in the central portion are connected by the first pattern connection portions that connect the one end side apexes and the connection linear portions located closer to one end side than the one end side apexes, and the fourth pattern connection portions that connect the one end side apexes and the other end side apexes, and the first pattern connection portions and the fourth pattern connection portions are alternately arranged in a circumferential direction.

9. The in-vivo indwelling stent according to claim 1, wherein all the adjacent annular bodies located in the central portion are connected by the first pattern connection portions that connect the one end side apexes and the connection linear portions located closer to one end side than the one end side apexes, and the fourth pattern connection portions that connect the one end side apexes and the other end side apexes located closer to one end side than the one end side apexes, and the first pattern connection portions and the fourth pattern connection portions are alternately arranged in a circumferential direction.

10. The in-vivo indwelling stent according to claim 1, wherein the connection portions adjacent to each other in the axial direction of the stent in all the adjacent annular bodies located in the central portion extend in different circumferential directions.

11. The in-vivo indwelling stent according to claim 1, wherein the connection portions of the same pattern adjacent to each other in the axial direction of the stent located in the central portion extend in different circumferential directions.

12. The in-vivo indwelling stent according to claim 4, wherein the stent includes:
one end portion annular body connection portions that connect other end side bent portions having the other end side apexes of the annular body located at one end and one end side bent portions having the one end side apexes of the annular body adjacent to the annular body located at the one end; and
other end portion annular body connection portions that connect one end side bent portions having the one end side apexes of the annular body located at the other end and other end side bent portions having the other end side apexes of the annular body adjacent to the annular body located at the other end, the one end portion annular body connection portions include the fourth pattern connection portions, and the other end portion annular body connection portions include the fourth pattern connection portions.

13. The in-vivo indwelling stent according to claim 1, wherein the connection portions are inclined in the axial direction of the stent, and the connection portions that connect the adjacent annular bodies are all inclined in the same direction.

14. The in-vivo indwelling stent according to claim 1, further comprising:
a plurality of convex portions on an outer surface of the stent.

15. The in-vivo indwelling stent according to claim 14, wherein a height of the convex portions is equal to or greater than 50 μm.

16. The in-vivo indwelling stent according to claim 1, wherein the stent is configured to be formed in a substantially cylindrical shape, to be compressed in a central axis direction at the time of in-vivo insertion, and to expand outward to restore a shape before compression at the time of in-vivo indwelling.

17. A stent delivery system comprising:
a sheath;
the stent according to claim 16 configured to be stored in a distal end portion of the sheath; and
a shaft configured to be inserted into the sheath and to release the stent from a distal end of the sheath.

18. An in-vivo indwelling stent comprising:
a plurality of annular bodies formed in a circular shape with linear components arranged in an axial direction, and adjacent annular bodies of the plurality of annual bodies connected by connection portions;
each of the plurality of annular bodies being formed with one endless linear component having a plurality of one end side apexes located on one end side in the axial direction of the stent, a plurality of other end side apexes located on the other end side in the axial direction of the stent, and a plurality of connection linear portions that connect the one end side apexes and the other end side apexes; and
the adjacent annular bodies including at least a central portion of the stent being connected by a plurality of connection portions, the plurality of connection portions located in the central portion being basic pattern connection portions including first pattern connection portions that connect the one end side apexes and the connection linear portions located closer to one end side than the one end side apexes or second pattern connection portions that connect the connection linear portions and the connection linear portions located closer to the other end side than the connection linear portions, and remaining connection portions of the plurality of connection portions located in the central portion being pattern connection portions of at least one type selected from the first pattern connection portions or the second pattern connection portions which are not selected in the basic pattern connection portions, third pattern connection portions that connect the other end side apexes and the connection linear portions located closer to the other end side than the other end side apexes, and fourth pattern connection portions that connect the one end side apexes and the other end side apexes located closer to one end side than the one end side apexes, and the number of connection portions that connect the adjacent annular bodies located in the central portion being the same as, larger by one than, or smaller by one than the number of the one end side apexes or the number of the other end side apexes of the annular bodies connected by the connection portions.

19. The in-vivo indwelling stent according to claim 18, wherein the adjacent annular bodies located in the central portion are connected by the first pattern connection portions that connect the one end side apexes and the connection linear portions located closer to one end side than the one end side apexes, and the third pattern connection portions that connect the other end side apexes and the connection linear portions located closer to the other end side than the other end side apexes, and the first pattern connection portions and the third pattern connection portions are alternately arranged in a circumferential direction between the adjacent annular bodies.

20. An in-vivo indwelling stent comprising:
a plurality of annular bodies formed with linear components, each of the plurality of annular bodies being formed with one endless linear component having a plurality of one end side apexes located on one end side in the axial direction of the stent, a plurality of other end side apexes located on the other end side in the axial direction of the stent, and a plurality of connection linear portions that connect the one end side apexes and the other end side apexes;
a plurality of connection portions, the plurality of connection portions including basic pattern connection portions and other connection portions, the basic pattern connection including first pattern connection portions that connect one end side apexes and connection linear portions located closer to one end side than the one end side apexes or second pattern connection portions that connect the connection linear portions and the connection linear portions closer to the other end side than the connection linear portions, and the other connection portions being selected from the first or second pattern connection portions which are not selected, third pattern connection portions that connect the other end side apexes and the connection linear portions closer to the other end side than the other end side apexes, and fourth pattern connection portions that connect the one end side apexes and the other end side apexes located closer to one end side than the one end side apexes; and
wherein a number of the connection portions between adjacent annular bodies is the same as, larger by one than, or smaller by one than the number of the one end side apexes or the other end side apexes of the annular bodies to be connected.
